SEQUENCE LISTING (134 sequences)

SEQ ID NO: 1 - DNA, 30 bp, Homo sapiens
atgctcaaggattgagcataactatacaac

SEQ ID NO: 2 - DNA, 30 bp, Homo sapiens
ctactacctcatacgagttaggaactcgta

SEQ ID NO: 3 - RNA, 22 nt, Homo sapiens
ugagguaguagguuguauaguu

SEQ ID NO: 4 - DNA, 16 bp, Homo sapiens
tgttggatgatggagt

SEQ ID NO: 5 - DNA, 41 bp, Homo sapiens
gtgctcaaggattgagcacaactatacaacctactacctca

SEQ ID NO: 6 - RNA, 21 nt, Homo sapiens
ucccugagaccucaaguguga

SEQ ID NO: 7 - DNA, 16 bp, Homo sapiens
gaactccagagtccct

SEQ ID NO: 8 - DNA, 40 bp, Homo sapiens
gtgctcaaggattgagcactcacacttgaggtctcaggga

SEQ ID NO: 9 - RNA, 22 nt, Homo sapiens
uaaagugcuuauagugcaggua

SEQ ID NO: 10 - DNA, 16 bp, Homo sapiens
gtgatattcgtgaaat

SEQ ID NO: 11 - DNA, 41 bp, Homo sapiens
gtgctcaaggattgagcactacctgcactataagcacttta

SEQ ID NO: 12 - RNA, 22 nt, Homo sapiens
cuuucagucggauguuugcagc

SEQ ID NO: 13 - DNA, 41 bp, Homo sapiens
gtgctcaaggattgagcacgctgcaaacatccgactgaaag

SEQ ID NO: 14 - RNA, 21 nt, Homo sapiens
uggaagacuagugauuuuguu

SEQ ID NO: 15 - DNA, 16 bp, Homo sapiens
ttagtgatcagaaggt

SEQ ID NO: 16 - DNA, 40 bp, Homo sapiens
gtgctcaaggattgagcacaacaaaatcactagtcttcca

SEQ ID NO: 17 - RNA, 23 nt, Homo sapiens
agcagcauuguacagggcuauca

SEQ ID NO: 18 - DNA, 16 bp, Homo sapiens
ggacatgttacgacga

SEQ ID NO: 19 - DNA, 42 bp, Homo sapiens
gtgctcaaggattgagcactgatagccctgtacaatgctgct

SEQ ID NO: 20 - RNA, 21 nt, Homo sapiens
uuuggauugaagggagcucua

SEQ ID NO: 21 - DNA, 16 bp, Homo sapiens
gagggaagttaggttt

SEQ ID NO: 22 - DNA, 40 bp, Homo sapiens
gtgctcaaggattgagcactagagctcccttcaatccaaa

SEQ ID NO: 23 - RNA, 21 nt, Homo sapiens
uugaaagugacuacaucgggg

SEQ ID NO: 24 - DNA, 16 bp, Homo sapiens
tacatcagtgaaagtt

SEQ ID NO: 25 - DNA, 40 bp, Homo sapiens
gtgctcaaggattgagcacccccgatgtagtcactttcaa

SEQ ID NO: 26 - RNA, 23 nt, Homo sapiens
uaaggcacgcggugaaugccaag

SEQ ID NO: 27 - DNA, 16 bp, Homo sapiens
aagtggcgcacggaat

SEQ ID NO: 28 - DNA, 42 bp, Homo sapiens
gtgctcaaggattgagcaccttggcattcaccgcgtgcctta

SEQ ID NO: 29 - RNA, 21 nt, Homo sapiens
uugugcgugugacagcggcua

SEQ ID NO: 30 - DNA, 16 bp, Homo sapiens
cgacagtgtgcgtgtt

SEQ ID NO: 31 - DNA, 40 bp, Homo sapiens
gtgctcaaggattgagcactagccgctgtcacacgcacaa

SEQ ID NO: 32 - RNA, 23 nt, Homo sapiens
uaucacagccagcuuugaugugc

SEQ ID NO: 33 - DNA, 16 bp, Homo sapiens
tttcgaccgacactat

SEQ ID NO: 34 - DNA, 42 bp, Homo sapiens
gtgctcaaggattgagcacgcacatcaaagctggctgtgata

SEQ ID NO: 35 - RNA, 22 nt, Homo sapiens
uagcagcacguaaauauuggcg

SEQ ID NO: 36 - DNA, 16 bp, Homo sapiens
ataaatgcacgacgat

SEQ ID NO: 37 - DNA, 41 bp, Homo sapiens
gtgctcaaggattgagcaccgccaatatttacgtgctgcta

SEQ ID NO: 38 - RNA, 22 nt, Homo sapiens
uagcuuaucagacugauguuga

SEQ ID NO: 39 - DNA, 16 bp, Homo sapiens
agtcagactattcgat

SEQ ID NO: 40 - DNA, 41 bp, Homo sapiens
gtgctcaaggattgagcactcaacatcagtctgataagcta

SEQ ID NO: 41 - RNA, 22 nt, Homo sapiens
aagcugccaguugaagaacugu

SEQ ID NO: 42 - DNA, 16 bp, Homo sapiens
gaagttgaccgtcgaa

SEQ ID NO: 43 - DNA, 41 bp, Homo sapiens
gtgctcaaggattgagcacacagttcttcaactggcagctt

SEQ ID NO: 44 - RNA, 22 nt, Homo sapiens
uucaaguaauccaggauaggcu

SEQ ID NO: 45 - DNA, 16 bp, Homo sapiens
aggacctaatgaactt

SEQ ID NO: 46 - DNA, 41 bp, Homo sapiens
gtgctcaaggattgagcacagcctatcctggattacttgaa

SEQ ID NO: 47 - RNA, 22 nt, Homo sapiens
cuagcaccaucugaaaucgguu

SEQ ID NO: 48 - DNA, 16 bp, Homo sapiens
aaagtctaccacgatc

SEQ ID NO: 49 - DNA, 41 bp, Homo sapiens
gtgctcaaggattgagcacaaccgatttcagatggtgctag

SEQ ID NO: 50 - RNA, 22 nt, Homo sapiens
uggcagugucuuagcugguugu

SEQ ID NO: 51 - DNA, 16 bp, Homo sapiens
tcgattctgtgacggt

SEQ ID NO: 52 - DNA, 41 bp, Homo sapiens
gtgctcaaggattgagcacacaaccagctaagacactgcca

SEQ ID NO: 53 - RNA, 23 nt, Homo sapiens
aacauucaacgcugucggugagu

SEQ ID NO: 54 - DNA, 16 bp, Homo sapiens
ctgtcgcaacttacaa

SEQ ID NO: 55 - DNA, 42 bp, Homo sapiens
gtgctcaaggattgagcacactcaccgacagcgttgaatgtt

SEQ ID NO: 56 - RNA, 24 nt, Homo sapiens
cucuaauacugccugguaaugaug

SEQ ID NO: 57 - DNA, 16 bp, Homo sapiens
ggtccgtcataatctc

SEQ ID NO: 58 - DNA, 43 bp, Homo sapiens
gtgctcaaggattgagcaccatcattaccaggcagtattagag

SEQ ID NO: 59 - RNA, 21 nt, Homo sapiens
ugucaguuugucaaauacccc

SEQ ID NO: 60 - DNA, 16 bp, Homo sapiens
taaactgtttgactgt

SEQ ID NO: 61 - DNA, 40 bp, Homo sapiens
gtgctcaaggattgagcacggggtatttgacaaactgaca

SEQ ID NO: 62 - RNA, 23 nt, Homo sapiens
caagucacuagugguuccguuua

SEQ ID NO: 63 - DNA, 16 bp, Homo sapiens
ttggtgatcactgaac

SEQ ID NO: 64 - DNA, 42 bp, Homo sapiens
gtgctcaaggattgagcactaaacggaaccactagtgacttg

SEQ ID NO: 65 - RNA, 22 nt, Homo sapiens
gcacauuacacggucgaccucu

SEQ ID NO: 66 - DNA, 16 bp, Homo sapiens
gctggcacattacacg

SEQ ID NO: 67 - DNA, 41 bp, Homo sapiens
gtgctcaaggattgagcacagaggtcgaccgtgtaatgtgc

SEQ ID NO: 68 - RNA, 23 nt, Homo sapiens
cgcauccccuagggcauuggugu

SEQ ID NO: 69 - DNA, 16 bp, Homo sapiens
acgggatcccctacgc

SEQ ID NO: 70 - DNA, 42 bp, Homo sapiens
gtgctcaaggattgagcacacaccaatgccctaggggatgcg

SEQ ID NO: 71 - RNA, 22 nt, Homo sapiens
cuggcccucucugcccuuccgu

SEQ ID NO: 72 - DNA, 16 bp, Homo sapiens
cccgtctctcccggtc

SEQ ID NO: 73 - DNA, 41 bp, Homo sapiens
gtgctcaaggattgagcacacggaagggcagagagggccag

SEQ ID NO: 74 - RNA, 23 nt, Homo sapiens
uacccuguagauccgaauuugug

SEQ ID NO: 75 - DNA, 16 bp, Homo sapiens
agcctagatgtcccat

SEQ ID NO: 76 - DNA, 42 bp, Homo sapiens
gtgctcaaggattgagcaccacaaattcggatctacagggta

SEQ ID NO: 77 - RNA, 22 nt, Homo sapiens
uacccuguagaaccgaauuugu

SEQ ID NO: 78 - DNA, 16 bp, Homo sapiens
agccaagatgtcccat

SEQ ID NO: 79 - DNA, 41 bp, Homo sapiens
gtgctcaaggattgagcacacaaattcggttctacagggta

SEQ ID NO: 80 - RNA, 21 nt, Homo sapiens
aucacauugccagggauuucc

SEQ ID NO: 81 - DNA, 16 bp, Homo sapiens
agggaccgttacacta

SEQ ID NO: 82 - DNA, 40 bp, Homo sapiens
gtgctcaaggattgagcacggaaatccctggcaatgtgat

SEQ ID NO: 83 - RNA, 22 nt, Homo sapiens
uucacaguggcuaaguuccgcc

SEQ ID NO: 84 - DNA, 16 bp, Homo sapiens
tgaatcggtgacactt

SEQ ID NO: 85 - DNA, 41 bp, Homo sapiens
gtgctcaaggattgagcacggcggaacttagccactgtgaa

SEQ ID NO: 86 - RNA, 23 nt, Homo sapiens
uguaaacauccuacacucucagc

SEQ ID NO: 87 - DNA, 16 bp, Homo sapiens
cacatcctacaaatgt

SEQ ID NO: 88 - DNA, 42 bp, Homo sapiens
gtgctcaaggattgagcacgctgagagtgtaggatgtttaca

SEQ ID NO: 89 - RNA, 22 nt, Homo sapiens
ugagaugaagcacuguagcuca

SEQ ID NO: 90 - DNA, 16 bp, Homo sapiens
tgtcacgaagtagagt

SEQ ID NO: 91 - DNA, 41 bp, Homo sapiens
gtgctcaaggattgagcactgagctacagtgcttcatctca

SEQ ID NO: 92 - RNA, 24 nt, Homo sapiens
guccaguuuucccaggaaucccuu

SEQ ID NO: 93 - DNA, 16 bp, Homo sapiens
ggacccttttgacctg

SEQ ID NO: 94 - DNA, 43 bp, Homo sapiens
gtgctcaaggattgagcacaagggattcctgggaaaactggac

SEQ ID NO: 95 - RNA, 21 nt, Homo sapiens
uagguaguuucauguuguugg

SEQ ID NO: 96 - DNA, 16 bp, Homo sapiens
ttgtactttgatggat

SEQ ID NO: 97 - DNA, 40 bp, Homo sapiens
gtgctcaaggattgagcacccaacaacatgaaactaccta

SEQ ID NO: 98 - RNA, 21 nt, Homo sapiens
uaaucucagcuggcaacugug

SEQ ID NO: 99 - DNA, 16 bp, Homo sapiens
aacggtcgactctaat

SEQ ID NO: 100 - DNA, 40 bp, Homo sapiens
gtgctcaaggattgagcaccacagttgccagctgagatta

SEQ ID NO: 101 - DNA, 60 bp, Homo sapiens
gtgctcaaggattgagcacaactatacaacctactacctcagtgctcaaggattgagcac

SEQ ID NO: 102 - DNA, 59 bp, Homo sapiens
gtgctcaaggattgagcactcacacttgaggtctcagggagtgctcaaggattgagcac

SEQ ID NO: 103 - DNA, 60 bp, Homo sapiens
gtgctcaaggattgagcactacctgcactataagcactttagtgctcaaggattgagcac

SEQ ID NO: 104 - DNA, 60 bp, Homo sapiens
gtgctcaaggattgagcacgctgcaaacatccgactgaaaggtgctcaaggattgagcac

SEQ ID NO: 105 - DNA, 59 bp, Homo sapiens
gtgctcaaggattgagcacaacaaaatcactagtcttccagtgctcaaggattgagcac

SEQ ID NO: 106 - DNA, 61 bp, Homo sapiens
gtgctcaaggattgagcactgatagccctgtacaatgctgctgtgctcaaggattgagcac

SEQ ID NO: 107 - DNA, 59 bp, Homo sapiens
gtgctcaaggattgagcactagagctcccttcaatccaaagtgctcaaggattgagcac

SEQ ID NO: 108 - DNA, 59 bp, Homo sapiens
gtgctcaaggattgagcacccccgatgtagtcactttcaagtgctcaaggattgagcac

SEQ ID NO: 109 - DNA, 61 bp, Homo sapiens
gtgctcaaggattgagcaccttggcattcaccgcgtgccttagtgctcaaggattgagcac

SEQ ID NO: 110 - DNA, 59 bp, Homo sapiens
gtgctcaaggattgagcactagccgctgtcacacgcacaagtgctcaaggattgagcac

SEQ ID NO: 111 - DNA, 61 bp, Homo sapiens
gtgctcaaggattgagcacgcacatcaaagctggctgtgatagtgctcaaggattgagcac

SEQ ID NO: 112 - DNA, 60 bp, Homo sapiens
gtgctcaaggattgagcaccgccaatatttacgtgctgctagtgctcaaggattgagcac

SEQ ID NO: 113 - DNA, 60 bp, Homo sapiens
gtgctcaaggattgagcactcaacatcagtctgataagctagtgctcaaggattgagcac

SEQ ID NO: 114 - DNA, 60 bp, Homo sapiens
gtgctcaaggattgagcacacagttcttcaactggcagcttgtgctcaaggattgagcac

SEQ ID NO: 115 - DNA, 60 bp, Homo sapiens
gtgctcaaggattgagcacagcctatcctggattacttgaagtgctcaaggattgagcac

SEQ ID NO: 116 - DNA, 60 bp, Homo sapiens
gtgctcaaggattgagcacaaccgatttcagatggtgctaggtgctcaaggattgagcac

SEQ ID NO: 117 - DNA, 60 bp, Homo sapiens
gtgctcaaggattgagcacacaaccagctaagacactgccagtgctcaaggattgagcac

SEQ ID NO: 118 - DNA, 61 bp, Homo sapiens
gtgctcaaggattgagcacactcaccgacagcgttgaatgttgtgctcaaggattgagcac

SEQ ID NO: 119 - DNA, 62 bp, Homo sapiens
gtgctcaaggattgagcaccatcattaccaggcagtattagaggtgctcaaggattgagcac

SEQ ID NO: 120 - DNA, 59 bp, Homo sapiens
gtgctcaaggattgagcacggggtatttgacaaactgacagtgctcaaggattgagcac

SEQ ID NO: 121 - DNA, 61 bp, Homo sapiens
gtgctcaaggattgagcactaaacggaaccactagtgacttggtgctcaaggattgagcac

SEQ ID NO: 122 - DNA, 60 bp, Homo sapiens
gtgctcaaggattgagcacagaggtcgaccgtgtaatgtgcgtgctcaaggattgagcac

SEQ ID NO: 123 - DNA, 61 bp, Homo sapiens
gtgctcaaggattgagcacacaccaatgccctaggggatgcggtgctcaaggattgagcac

SEQ ID NO: 124 - DNA, 60 bp, Homo sapiens
gtgctcaaggattgagcacacggaagggcagagagggccaggtgctcaaggattgagcac

SEQ ID NO: 125 - DNA, 61 bp, Homo sapiens
gtgctcaaggattgagcaccacaaattcggatctacagggtagtgctcaaggattgagcac

SEQ ID NO: 126 - DNA, 60 bp, Homo sapiens
gtgctcaaggattgagcacacaaattcggttctacagggtagtgctcaaggattgagcac

SEQ ID NO: 127 - DNA, 59 bp, Homo sapiens
gtgctcaaggattgagcacggaaatccctggcaatgtgatgtgctcaaggattgagcac

SEQ ID NO: 128 - DNA, 60 bp, Homo sapiens
gtgctcaaggattgagcacggcggaacttagccactgtgaagtgctcaaggattgagcac

SEQ ID NO: 129 - DNA, 61 bp, Homo sapiens
gtgctcaaggattgagcacgctgagagtgtaggatgtttacagtgctcaaggattgagcac

SEQ ID NO: 130 - DNA, 60 bp, Homo sapiens
gtgctcaaggattgagcactgagctacagtgcttcatctcagtgctcaaggattgagcac

SEQ ID NO: 131 - DNA, 62 bp, Homo sapiens
gtgctcaaggattgagcacaagggattcctgggaaaactggacgtgctcaaggattgagcac

SEQ ID NO: 132 - DNA, 59 bp, Homo sapiens
gtgctcaaggattgagcacccaacaacatgaaactacctagtgctcaaggattgagcac

SEQ ID NO: 133 - DNA, 59 bp, Homo sapiens
gtgctcaaggattgagcaccacagttgccagctgagattagtgctcaaggattgagcac

SEQ ID NO: 134 - DNA, 16 bp, Homo sapiens
ttgtaggctgactttc

(19) United States
(12) Patent Application Publication
(10) Pub. No.: US 2007/0015176 A1
(43) Pub. Date: Jan. 18, 2007

(54) Small nucleic acid detection probes and uses thereof

(75) Inventors: Kai Qin Lao, Pleasanton, CA (US); Neil A. Straus, Emeryville, CA (US)

Correspondence Address: MILA KASAN, PATENT DEPT.;APPLIED BIOSYSTEMS, 850 LINCOLN CENTRE DRIVE, FOSTER CITY, CA, 94404, US

(73) Assignee: Applera Corporation, Foster City, CA (US)

(21) Appl. No.: 11/354,618

(22) Filed: Feb. 14, 2006

Related U.S. Application Data
(60) Provisional application No. 60/654,154, filed on Feb. 18, 2005.

(51) Int. Cl. C12Q 1/68; C07H 21/04

(52) U.S. Cl. 435/6; 536/24.3

(57) ABSTRACT

The present teachings are directed to compositions, methods, and kits for detecting and quantitating small nucleic acid molecules, including small DNA molecules and small RNA molecules. The detector probes of the current teachings, including single-loop detector probes, double-loop detector probes, and bimolecular detector probes, are designed to selectively hybridize with a corresponding small nucleic acid target and to produce, under appropriate conditions, a detectable signal or a detectably different signal. The detector complexes of the current teachings comprise a detector probe comprising a first reporter group and a displaceable sequence comprising a second reporter group, wherein the displaceable sequence is hybridized to the detector probe. According to certain methods, detecting a small nucleic acid target comprises the target displacing the displaceable sequence of a detector complex to form a detector probe-small nucleic acid target duplex, illuminating the duplex with light of an appropriate wavelength, and determining the presence of a detectable fluorescent signal or the change in a detectable signal.

### CROSS-REFERENCE TO RELATED APPLICATIONS

This application claims the benefit of priority under 35 U.S.C. § 119(e) from U.S. Patent Application No. 60/654,154, filed Feb. 18, 2005, which is incorporated herein by reference.

### FIELD

The present teachings generally relate to methods, reagents, and kits for detecting and/or quantifying small nucleic acid molecules.

### INTRODUCTION

The recent discovery that small nucleic acid molecules play a role in cell regulation, including without limitation gene silencing and translational repression, has lead to a great interest in further study of these molecules. Small RNA molecules, for example but not limited to small interfering RNA (siRNA) and microRNA (miRNA), have been implicated in gene regulation, chromatin condensation, antiviral defense, suppression of transposon hopping, and genomic rearrangement. Methods and reagents for detecting and quantitating small nucleic acid molecules, including their respective intracellular localization and distribution, would further current and future research, diagnostic, and therapeutic efforts.

### SUMMARY

The present teachings are directed to compositions, methods, and kits for detecting and quantitating small nucleic acid molecules, including without limitation untranslated functional RNA, non-coding RNA (ncRNA), small non-messenger RNA (snmRNA), and small DNA molecules. The detector probes of the current teachings, including unlooped detector probes, single-loop detector probes, double-loop detector probes, and bimolecular detector probes, are designed to selectively hybridize with a corresponding small nucleic acid molecule and to produce, under appropriate conditions, a detectable signal or a detectably different signal. The detector complexes of the current teachings comprise a detector probe and a displaceable sequence that is hybridized to the detector probe. In some embodiments, a detector probe comprises a first reporter group and the displaceable sequence comprises a second reporter group. In some embodiments, a first probe component comprises a first reporter group and the corresponding second probe component comprises a second reporter group. In some embodiments, a reporter probe comprises a tether and a dye molecule. In some embodiments, a first reporter group comprises a fluorophore and a second reporter group comprises a quencher or a dark quencher.

According to certain methods, detecting a small nucleic acid target comprises the target displacing the displaceable sequence of a detector complex to form a detector probe-small nucleic acid target duplex, illuminating the duplex with light of an appropriate wavelength, and determining the presence of a detectable fluorescent signal or the change in a detectable signal, including without limitation a spectral shift. In some embodiments, the determining comprises quantitating the detectable fluorescence or the change in a detectable fluorescence. Certain of the disclosed methods comprise in situ hybridization. Some methods comprise single molecule detection. In some embodiments, a multiplicity of different small nucleic acid targets are detected using a multiplicity of different detector complexes and/or a multiplicity of different bimolecular probes. In some multiplex detection methods, a first detector probe species comprises one reporter group species, a second detector probe species comprises a different reporter group species, a third detector probe species comprises yet another different reporter group species, and so on.

Kits for performing certain of the instant methods are also disclosed. Certain kit embodiments include an unlooped detector probe, a single-loop detector probe, a double-loop detector probe, a bimolecular detector probe, or combinations thereof. In some embodiments, a detector probe and/or a component of a bimolecular probe comprises a fluorescent reporter group and/or a quencher. In some embodiments, kits comprise a multiplicity of different detector probes for detecting and/or quantitating a multiplicity of different small nucleic acid molecules. In some embodiments, kits comprise a detector complex of the present teachings. In some embodiments, kits comprise a multiplicity of different detector complexes for detecting and/or quantitating a multiplicity of different small nucleic acid molecules.

These and other features of the present teachings are set forth herein.

### DRAWINGS

The skilled artisan will understand that the drawings, described below, are for illustration purposes only. These figures are not intended to limit the scope of the present teachings in any way.

FIG. 1: depicts one embodiment of the current teachings comprising a single-loop detection probe including a fluorescent reporter group (“F”).

FIG. 2: depicts one embodiment of the current teachings comprising a double-loop detection probe including a fluorescent reporter group (“F”).

FIG. 3: depicts one embodiment of the current teachings comprising a single-loop detection probe including a reporter group comprising an intercalating dye (“D”) and a tether.

FIG. 4: depicts one embodiment of the current teachings comprising a double-loop detection probe including a reporter group comprising an intercalating dye (“D”) and a tether.

FIG. 5: depicts one embodiment of the current teachings comprising a bimolecular detector probe.

### DESCRIPTION OF EXEMPLARY EMBODIMENTS

It is to be understood that both the foregoing general description and the following detailed description are exemplary and explanatory only and are not intended to limit the scope of the current teachings. In this application, the use of the singular includes the plural unless specifically stated otherwise. For example, “a detector probe” means that more than one detector probe can be present; for example, at least two copies of a particular detector probe species, as well as two or more different detector probe species. Also, the use of “comprise”, “comprises”, “comprising”, “contain”, “contains”, “containing”, “include”, “includes”, and “including” are not intended to be limiting. The term “and/or” means that the term before and the term after can be taken together or separately. For illustration purposes, but not as a limitation, “X and/or Y” can mean “X” or “Y” or “X and Y”.

The section headings used herein are for organizational purposes only and are not to be construed as limiting the described subject matter in any way. All literature and similar materials cited in this application, including but not limited to, patents, patent applications, articles, books, treatises, and internet web pages are expressly incorporated by reference in their entirety for any purpose. In the event that one or more of the incorporated literature and similar materials differs from or contradicts this application, including but not limited to defined terms, term usage, described techniques, or the like, this application controls.

I. Definitions

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, or CAB. Continuing with this example, expressly included are combinations that contain repeats of one or more item or term, such as BB, AAA, AAB, BBC, AAABCCCC, CBBAAA, CABABB, and so forth. The skilled artisan will understand that typically there is no limit on the number of items or terms in any combination, unless otherwise apparent from the context.

The term “corresponding” as used herein refers to at least one specific relationship between the elements to which the term refers. For example, a single loop detector probe anneals with the corresponding small nucleic acid target; a first probe component and the corresponding second probe component of a bimolecular probe anneal with the corresponding small nucleic acid target; and so forth.

The terms “groove binder” and “minor groove binder” refer to small molecules that fit into the minor groove of double-stranded DNA, typically in a sequence specific manner. Generally, minor groove binders are long, flat molecules that can adopt a crescent-like shape and thus, fit snugly into the minor groove of a double helix, often displacing water. Minor groove binding molecules typically comprise several aromatic rings connected by bonds with torsional freedom, such as but not limited to, furan, benzene, or pyrrole rings. Exemplary minor groove binders include without limitation, antibiotics such as netropsin, distamycin, berenil, pentamidine and other aromatic diamidines, Hoechst 33258, SN 6999, aureolic anti-tumor drugs such as chromomycin and mithramycin, CC-1065, dihydrocyclopyrroloindole tripeptide (DPI₃), 1,2-dihydro-(3H)-pyrrolo[3,2-e]indole-7-carboxylate (CDPI₃), and related compounds and analogues. In certain embodiments, a minor groove binder is a element of a detector probe or of a probe component of a bimolecular detector probe, for example but not limited to, an element of the target-complementary binding portion. Detailed descriptions of minor groove binders can be found in, among other places, Nucleic Acids in Chemistry and Biology, 2d ed., Blackburn and Gait, eds., Oxford University Press, 1996 (“Blackburn and Gait”), particularly in section 8.3; Kumar et al., Nucl. Acids Res. 26:831-38, 1998; Kutyavin et al., Nucl. Acids Res. 28:655-61, 2000; Turner and Denny, Curr. Drug Targets 1:1-14, 2000; Kutyavin et al., Nucl. Acids Res. 25:3718-25,1997; Lukhtanov et al., Bioconjug. Chem. 7:564-7, 1996; Lukhtanov et al., Bioconjug. Chem. 6: 418-26, 1995; U.S. Pat. No. 6,426,408; and PCT Published Application No. WO 03/078450.

The terms “hybridizing” and “annealing”, including variations of these terms such as annealed, hybridization, anneal, hybridizes, and so forth, are used interchangeably and mean the nucleotide base-pairing interaction of one nucleic acid with another nucleic acid that results in the formation of a duplex, triplex, or other higher-ordered structure. The primary interaction is typically nucleotide base specific, e.g., A:T, A:U, and G:C, by Watson-Crick and Hoogsteen-type hydrogen bonding. In certain embodiments, base-stacking and hydrophobic interactions may also contribute to duplex stability. Conditions under which probes anneal to corresponding target sequences are well known in the art, e.g., as described in Nucleic Acid Hybridization, A Practical Approach, Hames and Higgins, eds., IRL Press, Washington, D.C. (1985) and Wetmur and Davidson, Mol. Biol. 31:349,1968. In general, whether such annealing takes place is influenced by, among other things, the length of the complementary portion of the probes and their corresponding targets or regions of targets, the pH, the temperature, the presence of mono- and divalent cations, the proportion of G and C nucleotides in the hybridizing region, the viscosity of the medium, and the presence of denaturants. Such variables influence the time required for hybridization. The presence of certain nucleotide analogs or groove binders in the complementary portions of the disclosed detector probes and/or components of detector probes can also influence hybridization conditions. Thus, the preferred annealing conditions will depend upon the particular application. Such conditions, however, can be routinely determined by persons of ordinary skill in the art, without undue experimentation. Typically, annealing conditions are selected to allow target-complementary portions of detector probes, detector probe subunits, and displaceable sequences to selectively hybridize with their corresponding target sequence or a subsequence of a target-complementary portion of a corresponding probe, respectively, but not hybridize to any significant degree to other sequences in the reaction.

The term “in situ hybridization” or “ISH”, as used herein refers to any process wherein a detector probe, including the subunits of a bimolecular detector probe, and/or a detector complex, is combined with a sample comprising a cell, including cells within a tissue, an embryo, or in a smear, such as a blood smear; the detector probe enters the cell and anneals with the corresponding small nucleic acid target, for example but not limited to a miRNA or a siRNA; and the presence of the detector probe-small nucleic acid molecule duplex or trimolecular complex (i.e., the two subunits of a bimolecular detector probe annealed with corresponding regions of the small nucleic acid target) can be detected in the whole mount, tissue section or cell by, for example, fluorescence microscopy. In some embodiments, the sample is morphologically preserved or is still living. In some embodiments, detecting comprises quantitative image analysis techniques. In some embodiments, a tissue is sectioned from a paraffin-embedded or frozen tissue and the section is fixed on a substrate, for example, a glass slide. In some embodiments, a cell is fixed on a substrate, for example cells grown on the substrate and then fixed, or cells in a smear that are spread on a substrate and then fixed, including without limitation drying and/or heating. In some embodiments, cells are grown on a substrate and then probed without fixation other than cell adhesion to the substrate, for example but not limited to a suitable tissue culture vessel or cover slip, or the cells can be cytospun onto the substrate. In some embodiments, combining a detector probe or a detector complex with a cell comprises microinjection, a vesicle, which may but need not comprise a liposome, or other transfection composition. Typically the fixation methods employed are very mild or gentle to minimize the loss of small nucleic acid sequences from the section or cell. Those in the art will appreciate that the disclosed detector complexes that comprise displaceable sequences containing a dark quencher typically produces little or no fluorescence, so extensive washing steps are not necessary and are typically omitted to minimize target loss.

An intercalating dye molecule, including for the purposes of the current teachings, groove binding dyes, is any of a variety of dye compounds that detectably interact with double-stranded DNA, preferably in a double-strand specific manner or at least to a measurably higher degree. Exemplary intercalating dyes (including minor groove binding dyes) for detecting double-stranded DNA include: ethidium bromide, BEBO (Bengtsson et al., Nucl. Acids Res 31:e45, 2003), EnhanCE™ (Beckman Coulter), Hoechst 33258 (bis-benzimide), Hoechst 33342, Hoechst 34580, DAPI (4′,6-diamidino-2-phenylindole), pyrylium iodide, PicoGreen, and SYBR Green I (Molecular Probes, Eugene Oreg.). Some detector probes of the current teachings contain an intercalating dye molecule on a “tether” or linker. A tether of the current teachings is typically a polymer that is often flexible, but not always, for example but not limited to a hydrocarbon chain such as polyethylene glycol. The tether keeps the dye molecule within an appropriate distance so that after the detector probe and small nucleic acid target hybridize, the dye molecule can intercalate in or bind in the groove of the resulting duplex and, under suitable illumination, produce a detectable signal or a detectable change in signal. The tether and dye molecule can be located on or near the ends of the detector probe or detector probe subunit, or it may be located internally. Those in the art will understand that the length and composition of the disclosed tether can vary depending, at least in part, on the specific dye molecule, but that appropriate tethers can be identified using routine methods and without undue experimentation (see, e.g., Almadidy et al., Can. J. Chem. 81:339-49, 2003; Jakeway and Krull, Can. J. Chem. 77:2083-87,1999; and Wiederholt et al., Bioconj. Chem. 8:119-26,1997).

The term “nucleotide analog” refers to a synthetic analog having modified nucleotide base portions, modified pentose portions, and/or modified phosphate portions, and, in the case of polynucleotides, modified internucleotide linkages (see, e.g., Scheit, Nucleotide Analogs, John Wiley, New York, 1980; Englisch, Angew. Chem. Int. Ed. Engl. 30:613-29, 1991; Agarwal, Protocols for Polynucleotides and Analogs, Humana Press, 1994; and S. Verma and F. Eckstein, Ann. Rev. Biochem. 67:99-134, 1998). Generally, modified phosphate portions comprise analogs of phosphate wherein the phosphorous atom is in the +5 oxidation state and one or more of the oxygen atoms is replaced with a non-oxygen moiety, e.g., sulfur. Exemplary phosphate analogs include but are not limited to phosphorothioate, phosphorodithioate, phosphoroselenoate, phosphorodiselenoate, phosphoroanilothioate, phosphoranilidate, phosphoramidate, boronophosphates, including associated counterions, e.g., H⁺, NH₄⁺, Na⁺, if such counterions are present. Exemplary modified nucleotide base portions include but are not limited to 5-methylcytosine (5mC), C-5 propynyl-C, C-5 propynyl-U, 2,6-diaminopurine (also known as 2-amino adenine or 2-amino-dA), hypoxanthine, pseudouridine, 2-thiopyrimidine, isocytosine (isoC), 5-methyl isoC, and isoguanine (isoG; see, e.g., U.S. Pat. No. 5,432,272). Exemplary modified pentose portions include but are not limited to, locked nucleic acid (LNA) analogs including without limitation Bz-A-LNA, 5-Me-Bz-C-LNA, dmf-G-LNA, and T-LNA (see, e.g., The Glen Report, 16(2):5, 2003; Koshkin et al., Tetrahedron 54:3607-30,1998), and 2′- or 3′-modifications where the 2′- or 3′-position is hydrogen, hydroxy, alkoxy (e.g., methoxy, ethoxy, allyloxy, isopropoxy, butoxy, isobutoxy and phenoxy), azido, amino, alkylamino, fluoro, chloro, or bromo. Modified internucleotide linkages include phosphate analogs, analogs having achiral and uncharged intersubunit linkages (e.g., Sterchak, E.P., et al., Organic Chem, 52:4202, 1987), and uncharged morpholino-based polymers having achiral intersubunit linkages (see, e.g., U.S. Pat. No. 5,034,506). Some internucleotide linkage analogs include morpholidate, acetal, and polyamide-linked heterocycles. In one class of nucleotide analogs, known as peptide nucleic acids, including pseudocomplementary peptide nucleic acids (“PNA”), a conventional sugar and internucleotide linkage has been replaced with a 2-aminoethylglycine amide backbone polymer (see, e.g., Nielsen et al., Science, 254:1497-1500, 1991; Egholm et al., J. Am. Chem. Soc., 114: 1895-1897 1992; Demidov et al., Proc. Natl. Acad. Sci. 99:5953-58, 2002; Peptide Nucleic Acids: Protocols and Applications, Nielsen, ed., Horizon Bioscience, 2004). A wide range of nucleotide analogs are available as triphosphates, phoshoramidites, or CPG derivatives for use in enzymatic incorporation or chemical synthesis from, among other sources, Glen Research, Sterling, Md.; Link Technologies, Lanarkshire, Scotland, UK; and TriLink BioTechnologies, San Diego, Calif. Descriptions of oligonucleotide synthesis and nucleotide analogs, can be found in, among other places, S. Verma and F. Eckstein, Ann. Rev. Biochem. 67:99-134, 1999; Goodchild, Bioconj. Chem. 1:165-87,1990; Current Protocols in Nucleic Acid Chemistry, Beaucage et al., eds., John Wiley & Sons, 1999, including supplements through February 2005 (“Beaucage”); and Blackburn and Gait.

The term “reporter group” is used in a broad sense herein and refers to any identifiable tag, label, or moiety. The skilled artisan will appreciate that many different species of reporter groups can be used in the present teachings, either individually or in combination with one or more different reporter group. The term reporter group also encompasses an element of multi-element indirect reporter systems, including without limitation, affinity tags; and multi-element interacting reporter groups or reporter group pairs, such as fluorescent reporter group-quencher pairs, including without limitation, fluorescent quenchers and dark quenchers, also known as non-fluorescent quenchers (NFQ). A fluorescent quencher can absorb the fluorescent signal emitted from a fluorophore and after absorbing enough fluorescent energy, the fluorescent quencher can emit fluorescence at a characteristic wavelength, e.g., fluorescent resonance energy transfer. For example without limitation, the FAM-TAMRA pair can be illuminated at 492 nm, the excitation peak for FAM, and emit fluorescence at 580 nm, the emission peak for TAMRA. A dark quencher, appropriately paired with a fluorescent reporter group, absorbs the fluorescent energy from the fluorophore, but does not itself fluoresce. Rather, the dark quencher dissipates the absorbed energy, typically as heat. Exemplary dark or nonfluorescent quenchers include Dabcyl, Black Hole Quenchers, Iowa Black, QSY-7, AbsoluteQuencher, Eclipse non-fluorescent quencher, certain metal particles such as gold nanoparticles, and the like.

In certain embodiments, a reporter group emits a fluorescent, a chemiluminescent, a bioluminescent, a phosphorescent, a radioactive, a calorimetric, or an electrochemiluminescent signal. Exemplary reporter groups include, but are not limited to fluorophores, radioisotopes, chromogens, enzymes, antigens including but not limited to epitope tags, semiconductor nanocrystals such as quantum dots, heavy metals, dyes, phosphorescence groups, chemiluminescent groups, electrochemical detection moieties, affinity tags, binding proteins, phosphors, rare earth chelates, transition metal chelates, near-infrared dyes, electrochemiluminescence labels, and the like.

The term reporter group also encompasses an element of multi-element reporter systems, including without limitation, affinity tags such as biotin:avidin, or antibody:antigen in which one element interacts with one or more other elements of the system in order to effect the potential for a detectable signal. Exemplary multi-element reporter systems include a detector probe comprising a biotin reporter group and a streptavidin-conjugated fluorophore or a bimolecular detector probe component comprising a DNP reporter group and a fluorophore-labeled anti-DNP antibody. Detailed protocols for attaching reporter groups to oligonucleotides, polynucleotides, peptides, antibodies and the like can be found in, among other places, G. T. Hermanson, Bioconjugate Techniques, Academic Press, San Diego, 1996 (“Hermanson”); Beaucage; R. Haugland, Handbook of Fluorescent Probes and Research Products, 9^{th} ed. (2002), Molecular Probes, Eugene, Oreg. (“Molecular Probes Handbook”); and Pierce Applications Handbook and Catalog 2003-2004, Pierce Biotechnology, Rockford, Ill., 2003 (“Pierce Applications Handbook”).

The terms “fluorophore” and “fluorescent reporter group” are intended to include any compound, label, or moiety that absorbs energy, typically from an illumination source, to reach an electronically excited state, and then emits energy, typically at a characteristic wavelength, to achieve a lower energy state. For example but without limitation, when certain fluorophores are illuminated by an energy source with an appropriate excitation wavelength, typically an incandescent or laser light source, photons in the fluorophore are emitted at a characteristic fluorescent emission wavelength. Fluorophores, sometimes referred to as fluorescent dyes, may typically be divided into families, such as fluorescein and its derivatives; rhodamine and its derivatives; cyanine and its derivatives; coumarin and its derivatives; Cascade Blue™ and its derivatives; Lucifer Yellow and its derivatives; BODIPY and its derivatives; and so forth. Exemplary fluorophores include indocarbocyanine (C3), indodicarbocyanine (C5), Cy3, Cy3.5, Cy5, Cy5.5, Cy7, Texas Red, Pacific Blue, Oregon Green 488, Alexa Fluor 488, Alexa Fluor 532, Alexa Fluor 546, Alexa Fluor 568, Alexa Fluor 594, Alexa Fluor 647, Alexa Fluor 660, Alexa Fluor 680, JOE, Lissamine, Rhodamine Green, BODIPY, fluorescein isothiocyanate (FITC), carboxy-fluorescein (FAM), phycoerythrin, rhodamine, dichlororhodamine (dRhodamine™), carboxy tetramethylrhodamine (TAMRA™), carboxy-X-rhodamine (ROX™), LIZTM, VICTM, NEDTM, PETTM, SYBR, PicoGreen, RiboGreen, and the like. Descriptions of fluorophores and their use, can be found in, among other places, Molecular Probes Handbook; M. Schena, Microarray Analysis (2003), John Wiley & Sons, Hoboken, N.J.; Synthetic Medicinal Chemistry 2003/2004 Catalog, Berry and Associates, Ann Arbor, Mich.; Hermanson; and Glen Research 2002 Catalog, Sterling, Va. Near-infrared dyes are expressly within the intended meaning of the terms fluorophore and fluorescent reporter group.

The term “sample” is used in a broad sense herein and is intended to include a wide range of biological materials, including without limitation cells, tissues including organs, and embryos, as well as compositions derived or extracted from such biological materials, including without limitation lysates, sonicates, and sections, such as tissue sections, an embryo section, or a whole mount embryo. Tissue culture cells, including explanted material, primary cells, secondary cell lines, and the like, as well as lysates, extracts, or materials obtained from any cells, are also within the meaning of the term sample as used herein. It will be appreciated that samples can be pre-treated to obtain fractions that are typically enriched for polynucleotides, including small nucleic acid molecules, using any of a variety of procedures known in the art, including commercially available kits and instruments, for example but not limited to, ABI PRISM® TransPrep System, BloodPrep Chemistry, NucPrep Chemistry, PrepMan Ultra Sample Preparation Reagent, ABI PRISM® 6100 Nucleic Acid PrepStation, ABI PRISM® 6700 Automated Nucleic Acid Workstation (all from Applied Biosystems), and the mirVana RNA isolation kit (Ambion, Austin, Tex.). Cells can also be lysed using known methods, for example by heating at 95° C. for 5 minutes, sonication, or in a lysis reagent, such as a Tris lysate buffer (e.g., 10 mM Tris-HCl, pH 8.0, 0.02% sodium azide, and 0.03% Tween-20) or a GuHCl lysis buffer (e.g., 2.5M GuHCl, 150 mM MES pH 6.0, 200 mM NaCl, 0.75% Tween-20), among others (see, e.g., U.S. Provisional Patent Application Ser. No. 60/643,180). All pre-treated biological materials, including without limitation, enriched fractions, lysates, and so forth are within the intended meaning of the term “sample”. Additionally, a sample can be from a human or from a non-human species, including without limitation, vertebrate species, for example but not limited to mouse, rat, hamster, dog, cat, pig, or various primate species; invertebrate species, for example but not limited to, Caenorhabditis elegans and Drosophila melanogaster; or plant species, for example but not limited to, Arabidopsis thaliana.

The term “single molecule detection” or “SMD” is used in a broad sense herein and refers to any technique or method that comprises individually detecting a molecular complex, for example but not limited to a trimolecular complex (comprising a small nucleic acid sequence and the first and second components of a bimolecular detector probe) and a detector probe-small nucleic acid target duplex. The term “individually detecting” as used herein refers to the process of evaluating and/or interrogating the reporter group species of separate, discrete molecular complexes, in contrast to ensemble detection of reporter group species in populations of molecular complexes, as routinely done, for example, in microarray or immunoassay techniques. In certain embodiments, individually detecting comprises optical detection of a molecular complex in solution. In certain embodiments, solution phase optical detection comprises timed-gated fluorescence. In certain embodiments, optical detection comprises an electrophoresis capillary, including without limitation, microcapillaries and nanocapillaries; a sheath flow; a microfluidic device; or combinations thereof, wherein molecular complexes are individually detected. In certain embodiments, individually detecting comprises detecting a molecular complex in a microdroplet. In certain embodiments, an electrodynamic trap is used to levitate at least one microdrop comprising a molecular complex. Detailed descriptions of SMD techniques for individually detecting a molecular complex in solution can be found in, among other places, Single Molecule Detection in Solution: Methods and Applications, C. Zander, J. Enderlein, and R. Keller, eds., John Wiley & Sons, Inc., 2002; M. Barnes et al., Anal. Chem. 67:A418-23, 1995; M. Barnes et al., J. Opt. Soc. Am. B 11:1297-1304, 1994; S. Nie and R. Zare, Ann. Rev. Biophys. Biomol. Struct. 26:567-96,1997; M. Foquet et al., Anal. Chem. 74:1415-22, 2002; S. Weiss, Science 283:1676-83, 1999; C. -Y. Kung et al., Anal. Chem. 70:658-661, 1998; M. Wabuyele et al., Electrophoresis 22:3939-3948, 2001; W. Ambrose et al., Chem. Rev. 99:2929-56,1999; P. Goodwin et al., Acc. Chem. Res. 29:607-13, 1996; and R. Keller et al., Anal. Chem. 74:316A-24A, 2002.

In certain embodiments, individually detecting comprises near field microscopy, including but not limited to near-field scanning optical microscopy; far-field microscopy, including but not limited to, far-field confocal microscopy and fluorescence-correlation spectroscopy; wide-field epi-illumination microscopy, evanescent wave excitation microscopy or total internal reflectance (TIR) microscopy; scanning confocal fluorescence microscopy; the multiparameter fluorescence detection (MFD) technique; two-photon excitation microscopy; or combinations thereof. In certain embodiments, individually detecting comprises fluorescence detection integrated with atomic-force microscopy, for example but not limited to, using an inverted optical microscope; or fluorescence excitation spectroscopy combined with shear-force microscopy. Detailed descriptions of such techniques can be found in, among other places, S. Nie and R. Zare, Ann. Rev. Biophys. Biomol. Struct. 26:567-96,1997; R. Brown et al., Review of Single Molecule Detection in Biological Applications, NPL Report COAM 2, National Physics Laboratory, Middlesex, United Kingdom, 2001; P. Rothwell et al., Proc. Natl. Acad. Sci. 100:1655-60, 2003; C. Eggeling et al., J. Biotechnol. 86:163-80, 2001; W. Ambrose et al., Chem. Rev. 99:2929-56, 1999; S. Weiss, Science 283:1676-83, 1999; G. Segers-Nolten et al., Nucl. Acid Res. 30:4720-27, 2002; and J. Michaelis et al., Nature 405:325-28, 2000.

A “small nucleic acid sequence”, “small nucleic acid target”, or “target”, as those terms are used herein, refers to a nucleotide sequence whose presence, absence, or quantity is being evaluated. A small nucleic acid target can comprise either DNA or RNA and may initially be either single-stranded or double-stranded. Those in the art will appreciate, however, that the disclosed detector probes and detector complexes anneal with single-stranded targets, including without limitation one strand of a double-stranded nucleic acid molecule. A small nucleic acid sequence of the current teachings is typically less than 200 nucleotides or base pairs, as appropriate and are preferably less than 100 nucleotides or base pairs long. In some embodiments, a target is approximately 70 nucleotides or base pairs long. In some embodiments, a target is less than 50 nucleotides of base pairs long, less than 30 nucleotides or base pairs long, less than 25 nucleotides or base pairs long, between 19 and 23 nucleotide or base pairs long, or 21-22 nucleotides or base pairs long. Exemplary small nucleic acid sequences include small DNA molecules and small RNA molecules, for example but not limited to certain non-coding DNA (ncDNA, sometimes referred to as non-protein-coding DNA; see, e.g., Bergman and Kreitman, Genome Res. 11:1335-45, 2001) and certain non-coding RNAs (ncRNAs), for example but not limited to, microRNA precursors (pre-miRNAs), microRNAs (miRNAs), small interfering RNAs (siRNAs), small nucleolar RNAs (snoRNAs), small nuclear RNAs (snRNAs), and spliceosomal RNA (see, e.g., S. Buckingham, Horizon Symposia: Understanding the RNAissance, May 2003, pp. 1-3, Nature Publishing).

II. Exemplary Embodiments

The detector probes of the present teachings are designed to specifically hybridize with a corresponding small nucleic acid molecule, but not other non-target or “background” nucleic acid molecules. In some embodiments, a detector probe includes a loop structure (e.g., a “stem-loop”) on one end. In other embodiments, a detector probe includes two loop structures. The bimolecular detector probes of the current teachings comprise a first probe component and a second probe component, either or both of which can comprise a loop structure. While not intending to be limited to a particular theory, such loop structures are believed to impart steric hindrance that impedes a longer, non-target nucleic acid molecule from mis-annealing with the detector probe, i.e., the loop structure(s) “cage” the end(s) of the target sequence that hybridizes with the detector probe, providing in essence, another level of specificity. Some embodiments of the disclosed detector probes and detector complexes do not comprise a loop structure.

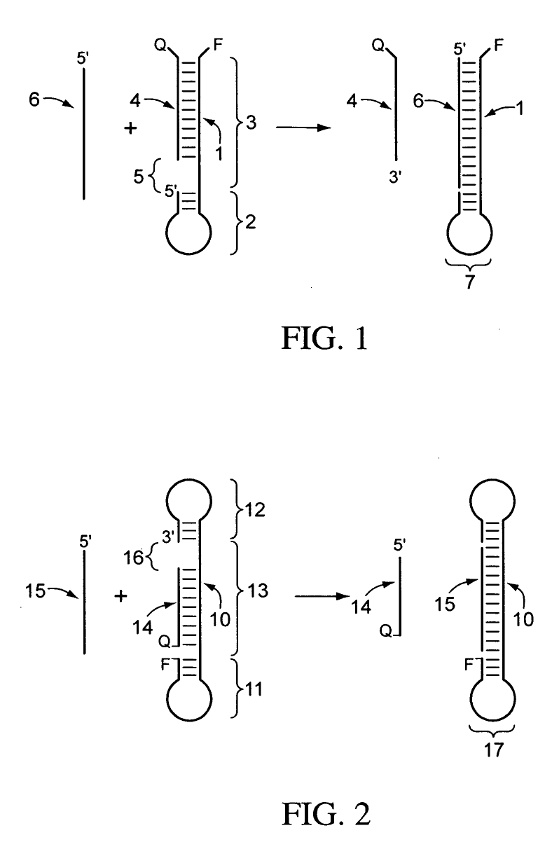

In some embodiments, a detector complex is provided that includes a detector probe and a displaceable sequence. The displaceable sequence is typically shorter than the target-complementary portion of the corresponding detector probe and is designed to anneal with a subsequence within the target-complementary portion of the detector probe. For illustration purposes but not as a limitation, for an exemplary target nucleic acid sequence that is 22 nucleotides long, the target-complementary portion of the corresponding detector probe is typically 22 nucleotides long, while the annealing portion of the displaceable sequence is typically shorter, for example 14, 16, or 18 nucleotides long. Since the displaceable sequence is shorter than the target-complementary portion of the detector probe, a gap exists at one or both ends of the detector complex. In some embodiments, the location of the gap is designed to be adjacent to the 3′-end of the displaceable sequence, e.g., as shown in FIG. 1. Those in the art will appreciate that in those embodiments in which the displaceable sequence is shorter than the small nucleic acid target, the displaceable sequence will have a lower Tm than the target, facilitating the displacement of the displaceable sequence from the corresponding detector complex and the formation of a detector probe-small nucleic acid target duplex at or near the annealing temperature of the target, above the Tm of the displaceable sequence.

In some embodiments, the detector probe comprises a first fluorescent reporter group and the displaceable sequence comprises a second reporter group. In some embodiments, the second reporter group comprises a fluorescent quencher or a dark quencher. In some embodiments, the first fluorescent reporter group and the second reporter group are selected to allow fluorescent resonant energy transfer (FRET) between the first fluorescent reporter group and the second reporter group when they are within an appropriate proximity of each other, for example but not limited to a FAM reporter group and a TAMRA reporter group (both available from Applied Biosystems).

In some embodiments, a bimolecular detector probe is provided, wherein the bimolecular probe comprise a first subunit and a second subunit. The first subunit comprises a first target-complementary portion, a first loop structure, and a first reporter group, wherein the first target-complementary portion is designed to anneal with a first region of a corresponding small nucleic acid molecule. The second subunit comprises a second target-complementary portion, a second loop structure, and a second reporter group, wherein the second target-complementary portion is designed to anneal with the second region of the same small nucleic acid molecule. In some embodiments, only one subunit of a bimolecular detector probe comprises a single loop structure. Typically, the first region of the small nucleic acid molecule and the second region of the small nucleic acid molecule are adjacent to each other. In some embodiments, the first reporter group comprises a first fluorescent reporter group and the second reporter group comprises a second fluorescent reporter group, wherein illumination of the first reporter group by light of an appropriate wavelength causes an energy transfer to the second reporter group and a fluorescent emission at a characteristic second wavelength.

In certain embodiments, the disclosed detector probes, bimolecular probe subunits, detector complexes, displaceable sequences, or combinations thereof, comprise nucleotide analogs to increase their resistance to nuclease degradation, relative to the same probe or sequence without such analogs, thereby enhancing their intracellular half-life, among other things. Exemplary analogs for such purposes include phosphorothioate deoxyribonucleotides, 2′-O-alkyl ribonucleotides, PNA, N3′-N5′ phosphoroamidites, 2′-deoxy-2′-fluoro-β-D-arabino nucleic acid (FANA), LNA, morpholino nucleotides, and cyclohexene nucleic acids (CeNA). Descriptions of such analogs can be found in, among other places, Kurreck, Eur. J. Biochem. 270:1628-44, 2003. Those in the art will appreciate that the incorporation of certain Tm enhancing nucleotide analogs in the disclosed probes or displaceable sequences may increase their Tm, which may create additional detector probe or displaceable sequence design issues, but that such issues can be resolved using routine skill and without undue experimentation. The term “Tm enhancing nucleotide analog” as used herein refers to a nucleotide analog that increases the melting temperature of a detector probe, a detector probe subunit, or a displaceable sequence of which it is a component, relative to a detector probe, a detector probe subunit, or a displaceable sequence with the same sequence comprising conventional nucleotides (A, C, G, and/or T), but not the Tm enhancing nucleotide analog. Exemplary Tm enhancing nucleotide analogs include C-5 propynyl-dC or 5-methyl-2′-deoxycytidine substituted for dC; 2,6-diaminopurine 2′-deoxyriboside (2-amino-dA) substituted for dA; and C-5 propynyl-dU for dT; which increase the relative melting temperature approximately 2.8° C., 1.3° C., 3.0° C., and 1.7° C. per substitution, respectively. Those in the art will appreciate that Tm can be determined experimentally using well-known methods or can be estimated using algorithms, thus one can readily determine whether a particular nucleotide analog will serve as a Tm enhancing nucleotide analog when used in a particular context, without undue experimentation.

The methods of the present teachings employ the disclosed detector probes and/or detector complexes to detect and to quantify small nucleic acid targets. According to certain methods, a detector complex is combined with a sample, wherein the detector complex comprises a detector probe annealed with a displaceable sequence. Under appropriate conditions, the displaceable sequence dissociates from the detector complex and is replaced by the corresponding small nucleic acid molecule to form a detector probe-small nucleic acid sequence duplex. Typically, the detector probe comprises a first fluorescent reporter group and the displaceable sequence comprises a second reporter group and the replacement of the displaceable sequence in the detector complex by the small nucleic acid target causes a detectable fluorescent signal or a detectable change in the fluorescent signal.

In one exemplary embodiment, shown in FIG. 1, a detector complex comprising (i) a detector probe 1, including a first loop structure 2, a target-complementary portion 3, and a first fluorescent reporter group (“F”), annealed with (ii) a displaceable sequence 4 comprising a second fluorescent reporter group (“Q”) and a gap 5 located between the 3′-end of the displaceable sequence 4 and the 5′-end of the first loop structure 2, is combined with a small nucleic acid target 6. The small nucleic acid target 6 replaces the displaceable sequence 4 in the detector complex (i.e., a detector probe-target duplex), causing the first fluorescent reporter group F and the second reporter group Q to dissociate, resulting in a detectable signal or a detectable change in signal. In some embodiments, the first reporter group is a fluorophore, such as Cy5, and the second reporter group is a dark quencher, such as Iowa Black, so that when the small nucleic acid molecule 6 replaces the displaceable sequence 4 a detectable signal is emitted when the complex is illuminated with light of the appropriate wavelength. In other embodiments, the first reporter group and the second reporter group form an interacting reporter group pair and when the two reporter groups are in appropriate proximity, FRET can occur. Thus, when the small nucleic acid molecule replaces the displaceable sequence 4, a change in detectable signal can be observed under appropriate illumination. In some embodiments, the fluorescent intensity can be quantitated and the amount of the small nucleic acid target can be inferred or calculated. In some embodiments, such quantitation comprises use of a standard or calibration curve.

In another exemplary embodiment, shown in FIG. 2, a detector complex comprising a double loop detector probe 10, comprising a first loop structure 11, a second loop structure 12, a target-complementary portion 13, and a first fluorescent reporter group (“F”), is initially annealed with a displaceable sequence 14 comprising a second fluorescent reporter group (“Q”). The gap 16 is located between the 5′-end of the displaceable sequence 14 and the 3′-end of the first loop structure 11. When combined with the corresponding small nucleic acid target 15, the displaceable sequence 14 is replaced by the small nucleic acid target 15 in the detector complex, causing the first fluorescent reporter group F and the second fluorescent reporter group Q to dissociate, resulting in a detectable signal or a detectable change in signal. Those in the art will appreciate that the loop structures on each end of the detector probe serve to limit the size of nucleic acid sequence that can anneal with the target complementary portion of the probe, thus reducing the possibility that a larger nucleic acid sequence such as a mRNA will mis-anneal with the probe. Those in the art will appreciate that the “gap” in the detector complex can be located between the 5′-end of the detector probe and the 3′-end of the displaceable sequence (see, e.g., FIG. 1), between the 3′-end of the detector probe and the 5′-end of the displaceable sequence (see, e.g., FIG. 2), or there can be a gap at both ends of the displaceable sequence relative to the detector probe to which it is annealed.

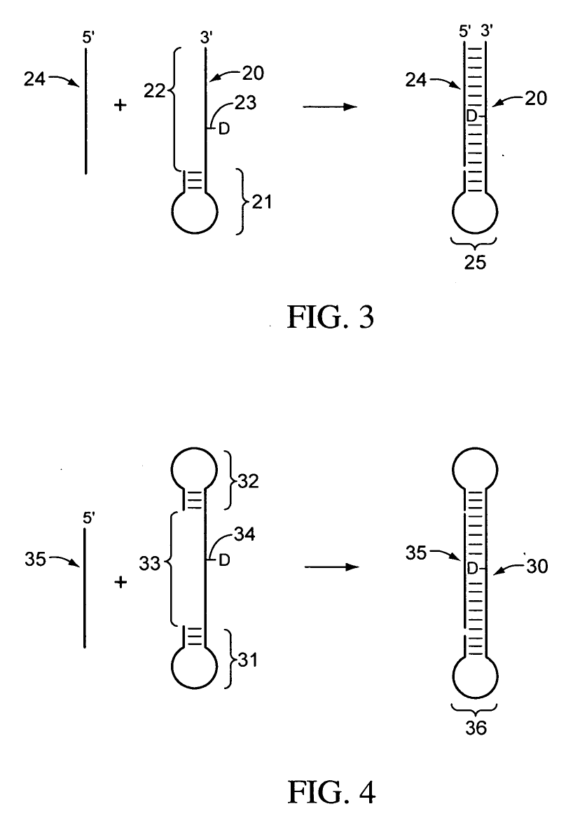

In another exemplary embodiment, shown in FIG. 3, a single loop detector probe 20 comprising a first loop structure 21, a target-complementary portion 22, and an intercalating dye molecule (“D”) on a tether 23 is combined with a small nucleic acid target 24. The small nucleic acid target 24 anneals with the target-complementary portion 22 of the probe 20, allowing the tethered dye D to intercalate into the detector probe-target sequence duplex 25 and, under appropriate illumination conditions, to emit a detectable signal or a change in a detectable signal, including without limitation a spectral shift.

In another exemplary embodiment, shown in FIG. 4, a two loop detector probe 30 comprising a first loop structure 31, a second loop structure 32, a target-complementary portion 33, and an intercalating dye molecule (“D”) on a tether 34, is combined with a corresponding small nucleic acid target 35. The small nucleic acid target 35 anneals with the target-complementary portion 33 of the probe 30, allowing the tethered dye molecule D to intercalate into the resulting detector probe-target sequence duplex 36 and, under appropriate illumination conditions, to emit a detectable signal or a change in a detectable signal, including without limitation a spectral shift.

In some embodiments, the gap sequence of the target-complementary portion of a detector probe comprises a minor groove binder to enhance the annealing of the corresponding small nucleic acid target without changing the Tm of the corresponding displaceable sequence. In some embodiments, a detector probe comprises a nucleotide analog. In some embodiments, the gap sequence of the target-complementary portion of a detector probe comprises a Tm enhancing nucleotide analog, to favor the annealing of the corresponding small nucleic acid target relative to the corresponding displaceable sequence. Exemplary Tm enhancing nucleotide analogs include 2,6-diaminopurine (2-amino-dA), 5-methylcytosine, C-5 propynyl-C, C-5 propynyl-U, locked nucleic acid (“LNA”, including without limitation, LNA-Bz-A, LNA-methyl-Bz-C, LNA-dmf-G, and LNA-T), and peptide nucleic acid (“PNA”, including without limitation pseudocomplementary PNA). Those in the art will understand that Tm enhancing nucleotide analogs can be identified using well known methods and without undue experimentation.

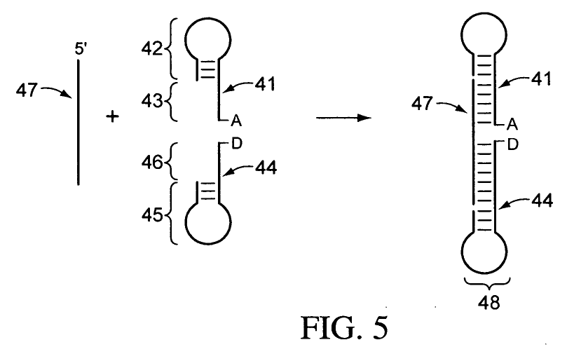

In another exemplary embodiment, shown in FIG. 5, a bimolecular detector probe comprising (i) a first probe component 41 that includes a first loop structure 42, a first target-complementary portion 43, and a first fluorescent reporter group (“A”) and (ii) a second probe component 44 that includes a second loop structure 45, a second target-complementary portion 46, and a second fluorescent reporter group (“D”), is combined with the corresponding small nucleic acid target 47. The small nucleic acid target 47 anneals with the first target-complementary portion 43 of the first probe component 41 and second target-complementary portion 46 of the second probe component 44 to form a trimolecular complex 48, wherein the first fluorescent reporter group A and the second fluorescent reporter group D are proximal to each other. Under appropriate illumination conditions, a detectable signal is emitted, including without limitation, a detectable change in signal due to FRET.

In some embodiments, the detector probes and/or detector complexes of the present teachings are useful for in situ hybridization detection and localization of small nucleic acid target. In some embodiments, tissue culture media, for example but not limited to serum-free media, comprising detector complexes is combined with living cells growing on an appropriate surface. The complexes are internalized, detector probe-target sequence duplexes form where appropriate, and the duplexes are detected using an appropriate detection means, such as fluorescence microscopy. In some embodiments, detecting includes an imaging/detection device such as a CCD camera, a CMOS camera, an avalanche photodiode, or a photomultiplier tube (PMT) and image processing software. In some embodiments, cells or tissue sections are fixed using mild fixation for example methanol and/acetic acid. The detector probe and/or detector complex in an appropriate hybridization solution, such as a 0.3 M NaCl solution, is combined with the fixed specimen and incubated. The presence of detectable signal or a change in a detectable signal is determined by fluorescent microscopy, typically with an associated imaging/detection device. In some ISH embodiments, detector probes and detector complexes are designed to produce little or no detectable signal prior to target hybridization, thus, extensive washing steps typically associated with conventional ISH protocols are typically unnecessary. Those in the art appreciate that, depending on whether the small nucleic acid target is RNA or DNA, appropriate RNase-free or DNase-free reagents, respectively should be employed when possible.

In some embodiments, a detector probe and/or a detector complex is combined with a sample, for example but not limited to a cell lysate, to form a reaction composition which is incubated under conditions suitable for detector probe-target sequence duplexes to form. In certain embodiments, the reaction composition is analyzed using a SMD technique, for example but not limited to a flow-through detection system, including without limitation a Trilogy™ Plafform (U.S. Genomics), that may include microfluidics, for example but not limited to microcapillaries and/or nanocapillaries; a biosensor; or other single molecule detection device, including without limitation attaching individual complexes to a capture surface followed by fluorescence detection or detecting individual complexes in a fluid flow (see, e.g., U.S. patent application Ser. No. 10/652430).

In some embodiments, detecting comprises quantitating the amount or relative amount of a small nucleic acid sequence or a multiplicity of different nucleic acid sequences in a sample. In some embodiments, a small nucleic acid target is quantitated by comparing the experimentally determined fluorescent intensity with a calibration or standard curve or by counting the number of fluorescent molecules per unit volume or per unit area. In some embodiments, a small nucleic acid target is quantitated using a SMD technique, for example but not limited to, counting the number of separated labeled duplexes per unit volume or per unit area. In some embodiments, a multiplicity of different small nucleic acid sequences are quantitated using a multiplicity of different detector complexes, wherein each species of detector complex comprises a different reporter group than any of the other species of detector complexes and the detectable emission or change in emission of each of the different reporter groups is quantified.

The instant teachings also provide kits designed to expedite performing the subject methods. Kits serve to expedite the performance of the methods of interest by assembling two or more components required for carrying out the methods. Kits preferably contain components in pre-measured unit amounts to minimize the need for measurements by end-users. Kits preferably include instructions for performing one or more of the disclosed methods. Preferably, the kit components are optimized to operate in conjunction with one another.

In certain embodiments, a kit comprises a single loop detector probe, a two loop detector probe, a single loop detector complex, a two loop detector complex, a displaceable sequence, a control sequence, or combinations thereof. Some embodiments comprise a multiplicity of different single loop detector probes, a multiplicity of different two loop detector probes, a multiplicity of different single loop detector complexes, a multiplicity of different two loop detector complexes, a multiplicity of different displaceable sequences, a multiplicity of different control sequences, or combinations thereof, for detecting a multiplicity of different small nucleic acid targets.

The current teachings, having been described above, may be better understood by reference to examples. The following examples are intended for illustration purposes only, and should not be construed as limiting the scope of the teachings herein in any way.

### EXAMPLE 1

Illustrative detector complexes comprising one loop or two loop detector probes and their corresponding small nucleic acid sequence targets.

Detector complexes comprising detector probes and their corresponding displaceable sequences can be synthesized using known techniques, based on the nucleotide sequence of the corresponding small nucleic acid targets. The sequences for some illustrative detector complexes comprising one loop detector probes and their corresponding small nucleic acid targets, human miRNAs in this example, are shown in Table 1. The sequences for some illustrative two loop detector probes and their corresponding small nucleic acid targets for detecting the same human miRNA targets as above, are shown in Table 2. The displaceable sequences shown in Table 1 are also used with the corresponding two loop detector probes in Table 2 to form illustrative detector complexes. The complementary sequences of the stem of each loop are shown in brackets and the loop segment is shown in italics for the first detector probe on each table.

### EXAMPLE 2

Trimolecular complex formation and detection in solution.

An exemplary bimolecular detector probe and a small nucleic acid target (let-7a1 miRNA) were synthesized. The first probe component comprised the sequence ATGCTCAAGGATTGAGCATAACTATACAAC-(TAMRA) (SEQ ID NO:1), including a first looped structure, comprising the complementary stem sequences (shown underlined) on either side of the loop sequence (shown in italics), a first target-complementary portion (no underline, no italics), and a first reporter group, TAMRA. The second probe component comprised the sequence (6-FAM)-CTACTACCTCATACGAGTTAGGAACTCGTA (SEQ ID NO:2), including a second looped structure, comprising the complementary stem sequences (shown underlined) on either side of the loop sequence (shown in italics), a second target-complementary portion (no underline, no italics), and a second reporter group, 6-FAM. The synthetic let-7a1 target sequence was ugagguaguagguuguauaguu (SEQ ID NO:3). A series of hybridization reactions were performed in parallel in wells of a 384 well plate. The first probe component, second probe component, and target were suspended in CHESS buffer (pH 9.0 at 37° C.). The first probe component and second probe component concentrations were 100 nM and the target concentrations were 40 nM, 8 nM, 1.6 nM, and 320 pM, respectively. ROX was used as a normalization dye in each reaction well. The plate was loaded into an ABI PRISM 7900HT Sequence Detection System instrument (Applied Biosystems) and the FAMFTAMRA fluorescence ratio was determined, as shown in Table 3.

TABLE 3

| | FAM/TAMRA |
|---|---|
| let-7a1 target | fluorescence |
| concentration | ratio |
| 40 nM | 2.67 |
| 8 nM | 4.07 |
| 1.6 nM | 4.43 |
| 320 pM | 5.02 |

Although the disclosed teachings has been described with reference to various applications, methods, and compositions, it will be appreciated that various changes and modifications may be made without departing from the teachings herein. The foregoing examples are provided to better illustrate the disclosed teachings and are not intended to limit the scope of the teachings herein.

TABLE 1

Illustrative detector complexes comprising single loop detector probes
and their corresponding displaceable probes and their respective small
nucleic acid target sequences.

| Target Sequence | | |
|---|---|---|
| Target Name | Displaceable Sequence | One Loop Detector Probe |
| ugagguaguagguuguauaguu | TGTTGGATGATGGAGT(IB) | [GTGCTCAA]GGA[TTGAGCAC]AACTATACAACCTACTACCTCA(Cy5) |
| let-7a1 (SEQ ID NO:3) | (SEQ ID NO:4) | (SEQ ID NO:5) |
| ucccugagaccucaaguguga | GAACTCCAGAGTCCCT(IB) | GTGCTCAAGGATTGAGCACTCACACTTGAGGTCTCAGGGA(Cy5) |
| lin-4 (SEQ ID NO:6) | (SEQ ID NO:7) | (SEQ ID NO:8) |
| uaaagugcuuauagugcaggua | GTGATATTCGTGAAAT(IB) | GTGCTCAAGGATTGAGCACTACCTGCACTATAAGCACTTTA(Cy5) |
| mir-20 (SEQ ID NO:9) | (SEQ ID NO:10) | (SEQ ID NO:11) |
| cuuucagucggauguuugcagc | TTGTAGGCTGACTTTC(IB) | GTGCTCAAGGATTGAGCACGCTGCAAACATCCGACTGAAAG(Cy5) |
| mir-30a (SEQ ID NO:12) | (SEQ ID NO:12) | (SEQ ID NO:13) |
| uggaagacuagugauuuuguu | TTAGTGATCAGAAGGT(IB) | GTGCTCAAGGATTGAGCACAACAAAATCACTAGTCTTCCA(Cy5) |
| mir-7 (SEQ ID NO:14) | (SEQ ID NO:15) | (SEQ ID NO:16) |
| agcagcauuguacagggcuauca | GGACATGTTACGACGA(IB) | GTGCTCAAGGATTGAGCACTGATAGCCCTGTACAATGCTGCT(Cy5) |
| mir-107 (SEQ ID NO:17) | (SEQ ID NO:18) | (SEQ ID NO:19) |
| uuuggauugaagggagcucua | GAGGGAAGTTAGGTTT(IB) | GTGCTCAAGGATTGAGCACTAGAGCTCCCTTCAATCCAAA(Cy5) |
| miR159a (SEQ ID NO:20) | (SEQ ID NO:21) | (SEQ ID NO:22) |
| uugaaagugacuacaucgggg | TACATCAGTGAAAGTT(IB) | GTGCTCAAGGATTGAGCACCCCCGATGTAGTCACTTTCAA(Cy5) |
| mir161 (SEQ ID NO:23) | (SEQ ID NO:24) | (SEQ ID NO:25) |
| uaaggcacgcggugaaugccaag | AAGTGGCGCACGGAAT(IB) | GTGCTCAAGGATTGAGCACCTTGGCATTCACCGCGTGCCTTA(Cy5) |
| mir-124(SEQ ID NO:26) | (SEQ ID NO:27) | (SEQ ID NO:28) |
| uugugcgugugacagcggcua | CGACAGTGTGCGTGTT(IB) | GTGCTCAAGGATTGAGCACTAGCCGCTGTCACACGCACAA(Cy5) |
| mir-210 (SEQ ID NO:29) | (SEQ ID NO:30) | (SEQ ID NO:31) |
| uaucacagccagcuuugaugugc | TTTCGACCGACACTAT(IB) | GTGCTCAAGGATTGAGCACGCACATCAAAGCTGGCTGTGATA(Cy5) |
| mir-2 (SEQ ID NO:32) | (SEQ ID NO:33) | (SEQ ID NO:34) |
| uagcagcacguaaauauuggcg | ATAAATGCACGACGAT(IB) | GTGCTCAAGGATTGAGCACCGCCAATATTTACGTGCTGCTA(Cy5) |
| mir-16 (SEQ ID NO:35) | (SEQ ID NO:36) | (SEQ ID NO:37) |
| uagcuuaucagacugauguuga | AGTCAGACTATTCGAT(IB) | GTGCTCAAGGATTGAGCACTCAACATCAGTCTGATAAGCTA(Cy5) |
| mir-21 (SEQ ID NO:38) | (SEQ ID NO:39) | (SEQ ID NO:40) |
| aagcugccaguugaagaacugu | GAAGTTGACCGTCGAA(IB) | GTGCTCAAGGATTGAGCACACAGTTCTTCAACTGGCAGCTT(Cy5) |
| mir-22 (SEQ ID NO:41) | (SEQ ID NO:42) | (SEQ ID NO:43) |
| uucaaguaauccaggauaggcu | AGGACCTAATGAACTT(IB) | GTGCTCAAGGATTGAGCACAGCCTATCCTGGATTACTTGAA(Cy5) |
| mir-26a (SEQ ID NO:44) | (SEQ ID NO:45) | (SEQ ID NO:46) |
| cuagcaccaucugaaaucgguu | AAAGTCTACCACGATC(IB) | GTGCTCAAGGATTGAGCACAACCGATTTCAGATGGTGCTAG(Cy5) |
| mir-29 (SEQ ID NO:47) | (SEQ ID NO:48) | (SEQ ID NO:49) |
| uggcagugucuuagcugguugu | TCGATTCTGTGACGGT(IB) | GTGCTCAAGGATTGAGCACACAACCAGCTAAGACACTGCCA(Cy5) |
| mir-34 (SEQ ID NO:50) | (SEQ ID NO:51) | (SEQ ID NO:52) |
| aacauucaacgcugucggugagu | CTGTCGCAACTTACAA(IB) | GTGCTCAAGGATTGAGCACACTCACCGACAGCGTTGAATGTT(Cy5) |
| mir-181a (SEQ ID NO:53) | (SEQ ID NO:54) | (SEQ ID NO:55) |
| cucuaauacugccugguaaugaug | GGTCCGTCATAATCTC(IB) | GTGCTCAAGGATTGAGCACCATCATTACCAGGCAGTATTAGAG(Cy5) |
| mir-200b (SEQ ID NO:56) | (SEQ ID NO:57) | (SEQ ID NO:58) |
| ugucaguuugucaaauacccc | TAAACTGTTTGACTGT(IB) | GTGCTCAAGGATTGAGCACGGGGTATTTGACAAACTGACA(Cy5) |
| mir-223 (SEQ ID NO:59) | (SEQ ID NO:60) | (SEQ ID NO:61) |
| caagucacuagugguuccguuua | TTGGTGATCACTGAAC(IB) | GTGCTCAAGGATTGAGCACTAAACGGAACCACTAGTGACTTG(Cy5) |
| mir-224 (SEQ ID NO:62) | (SEQ ID NO:63) | (SEQ ID NO:64) |
| gcacauuacacggucgaccucu | GCTGGCACATTACACG(IB) | GTGCTCAAGGATTGAGCACAGAGGTCGACCGTGTAATGTGC(Cy5) |
| mir-323 (SEQ ID NO:65) | (SEQ ID NO:66) | (SEQ ID NO:67) |
| cgcauccccuagggcauuggugu | ACGGGATCCCCTACGC(IB) | GTGCTCAAGGATTGAGCACACACCAATGCCCTAGGGGATGCG(Cy5) |
| mir-324-5 (SEQ ID NO:68) | (SEQ ID NO:69) | (SEQ ID NO:70) |
| cuggcccucucugcccuuccgu | CCCGTCTCTCCCGGTC(IB) | GTGCTCAAGGATTGAGCACACGGAAGGGCAGAGAGGGCCAG(Cy5) |
| mir-328 (SEQ ID NO:71) | (SEQ ID NO:72) | (SEQ ID NO:73) |
| uacccuguagauccgaauuugug | AGCCTAGATGTCCCAT(IB) | GTGCTCAAGGATTGAGCACCACAAATTCGGATCTACAGGGTA(Cy5) |
| mir-10a (SEQ ID NO:74) | (SEQ ID NO:75) | (SEQ ID NO:76) |
| uacccuguagaaccgaauuugu | AGCCAAGATGTCCCAT(IB) | GTGCTCAAGGATTGAGCACACAAATTCGGTTCTACAGGGTA(Cy5) |
| mir-10b (SEQ ID NO:77) | (SEQ ID NO:78) | (SEQ ID NO:79) |
| aucacauugccagggauuucc | AGGGACCGTTACACTA(IB) | GTGCTCAAGGATTGAGCACGGAAATCCCTGGCAATGTGAT(Cy5) |
| mir-23 (SEQ ID NO:80) | (SEQ ID NO:81) | (SEQ ID NO:82) |
| uucacaguggcuaaguuccgcc | TGAATCGGTGACACTT(IB) | GTGCTCAAGGATTGAGCACGGCGGAACTTAGCCACTGTGAA(Cy5) |
| mir-27 (SEQ ID NO:83) | (SEQ ID NO:84) | (SEQ ID NO:85) |
| uguaaacauccuacacucucagc | CACATCCTACAAATGT(IB) | GTGCTCAAGGATTGAGCACGCTGAGAGTGTAGGATGTTTACA(Cy5) |
| mir-30c (SEQ ID NO:86) | (SEQ ID NO:87) | (SEQ ID NO:88) |
| ugagaugaagcacuguagcuca | TGTCACGAAGTAGAGT(IB) | GTGCTCAAGGATTGAGCACTGAGCTACAGTGCTTCATCTCA(Cy5) |
| mir-143 (SEQ ID NO:89) | (SEQ ID NO:90) | (SEQ ID NO:91) |
| guccaguuuucccaggaaucccuu | GGACCCTTTTGACCTG(IB) | GTGCTCAAGGATTGAGCACAAGGGATTCCTGGGAAAACTGGAC(Cy5) |
| mir-145 (SEQ ID NO:92) | (SEQ ID NO:93) | (SEQ ID NO:94) |
| uagguaguuucauguuguugg | TTGTACTTTGATGGAT(IB) | GTGCTCAAGGATTGAGCACCCAACAACATGAAACTACCTA(Cy5) |
| mir-196 (SEQ ID NO:95) | (SEQ ID NO:96) | (SEQ ID NO:97) |
| uaaucucagcuggcaacugug | AACGGTCGACTCTAAT(IB) | GTGCTCAAGGATTGAGCACCACAGTTGCCAGCTGAGATTA(Cy5) |
| mir-216 (SEQ ID NO:98) | (SEQ ID NO:99) | (SEQ ID NO:100) |

TABLE 2
Illustrative two loop detector probes

| | |
|---|---|
| Target Name | Two Loop Detector Probe Sequence |

| | | |
|---|---|---|
| let-7a1 | [GTGCTCAA]GGA[TTGAGCAC]AACTATACAACCTACTACCTCA[GTGCTCAA]GGA[TTGAGCAC](Cy5) | (SEQ ID NO:101) |
| lin-4 | GTGCTCAAGGATTGAGCACTCACACTTGAGGTCTCAGGGAGTGCTCAAGGATTGAGCAC(Cy5) | (SEQ ID NO:102) |
| mir-20 | GTGCTCAAGGATTGAGCACTACCTGCACTATAAGCACTTTAGTGCTCAAGGATTGAGCAC(Cy5) | (SEQ ID NO:103) |
| mir-30a | GTGCTCAAGGATTGAGCACGCTGCAAACATCCGACTGAAAGGTGCTCAAGGATTGAGCAC(Cy5) | (SEQ ID NO:104) |
| mir-7 | GTGCTCAAGGATTGAGCACAACAAAATCACTAGTCTTCCAGTGCTCAAGGATTGAGCAC(Cy5) | (SEQ ID NO:105) |
| mir-107 | GTGCTCAAGGATTGAGCACTGATAGCCCTGTACAATGCTGCTGTGCTCAAGGATTGAGCAC(Cy5) | (SEQ ID NO:106) |
| miR159a | GTGCTCAAGGATTGAGCACTAGAGCTCCCTTCAATCCAAAGTGCTCAAGGATTGAGCAC(Cy5) | (SEQ ID NO:107) |
| mir161 | GTGCTCAAGGATTGAGCACCCCCGATGTAGTCACTTTCAAGTGCTCAAGGATTGAGCAC(Cy5) | (SEQ ID NO:108) |
| mir-124 | GTGCTCAAGGATTGAGCACCTTGGCATTCACCGCGTGCCTTAGTGCTCAAGGATTGAGCAC(Cy5) | (SEQ ID NO:109) |
| mir-210 | GTGCTCAAGGATTGAGCACTAGCCGCTGTCACACGCACAAGTGCTCAAGGATTGAGCAC(Cy5) | (SEQ ID NO:110) |
| mir-2 | GTGCTCAAGGATTGAGCACGCACATCAAAGCTGGCTGTGATAGTGCTCAAGGATTGAGCAC(Cy5) | (SEQ ID NO:111) |
| mir-16 | GTGCTCAAGGATTGAGCACCGCCAATATTTACGTGCTGCTAGTGCTCAAGGATTGAGCAC(Cy5) | (SEQ ID NO:112) |
| mir-21 | GTGCTCAAGGATTGAGCACTCAACATCAGTCTGATAAGCTAGTGCTCAAGGATTGAGCAC(Cy5) | (SEQ ID NO:113) |
| mir-22 | GTGCTCAAGGATTGAGCACACAGTTCTTCAACTGGCAGCTTGTGCTCAAGGATTGAGCAC(Cy5) | (SEQ ID NO:114) |
| mir-26a | GTGCTCAAGGATTGAGCACAGCCTATCCTGGATTACTTGAAGTGCTCAAGGATTGAGCAC(Cy5) | (SEQ ID NO:115) |
| mir-29 | GTGCTCAAGGATTGAGCACAACCGATTTCAGATGGTGCTAGGTGCTCAAGGATTGAGCAC(Cy5) | (SEQ ID NO:116) |
| mir-34 | GTGCTCAAGGATTGAGCACACAACCAGCTAAGACACTGCCAGTGCTCAAGGATTGAGCAC(Cy5) | (SEQ ID NO:117) |
| mir-181a | GTGCTCAAGGATTGAGCACACTCACCGACAGCGTTGAATGTTGTGCTCAAGGATTGAGCAC(Cy5) | (SEQ ID NO:118) |
| mir-200b | GTGCTCAAGGATTGAGCACCATCATTACCAGGCAGTATTAGAGGTGCTCAAGGATTGAGCAC(Cy5) | (SEQ ID NO:119) |
| mir-223 | GTGCTCAAGGATTGAGCACGGGGTATTTGACAAACTGACAGTGCTCAAGGATTGAGCAC(Cy5) | (SEQ ID NO:120) |
| mir-224 | GTGCTCAAGGATTGAGCACTAAACGGAACCACTAGTGACTTGGTGCTCAAGGATTGAGCAC(Cy5) | (SEQ ID NO:121) |
| mir-323 | GTGCTCAAGGATTGAGCACAGAGGTCGACCGTGTAATGTGCGTGCTCAAGGATTGAGCAC(Cy5) | (SEQ ID NO:122) |
| mir-324-5 | GTGCTCAAGGATTGAGCACACACCAATGCCCTAGGGGATGCGGTGCTCAAGGATTGAGCAC(Cy5) | (SEQ ID NO:123) |
| mir-328 | GTGCTCAAGGATTGAGCACACGGAAGGGCAGAGAGGGCCAGGTGCTCAAGGATTGAGCAC(Cy5) | (SEQ ID NO:124) |
| mir-10a | GTGCTCAAGGATTGAGCACCACAAATTCGGATCTACAGGGTAGTGCTCAAGGATTGAGCAC(Cy5) | (SEQ ID NO:125) |
| mir-10b | GTGCTCAAGGATTGAGCACACAAATTCGGTTCTACAGGGTAGTGCTCAAGGATTGAGCAC(Cy5) | (SEQ ID NO:126) |
| mir-23 | GTGCTCAAGGATTGAGCACGGAAATCCCTGGCAATGTGATGTGCTCAAGGATTGAGCAC(Cy5) | (SEQ ID NO:127) |
| mir-27 | GTGCTCAAGGATTGAGCACGGCGGAACTTAGCCACTGTGAAGTGCTCAAGGATTGAGCAC(Cy5) | (SEQ ID NO:128) |
| mir-30c | GTGCTCAAGGATTGAGCACGCTGAGAGTGTAGGATGTTTACAGTGCTCAAGGATTGAGCAC(Cy5) | (SEQ ID NO:129) |
| mir-143 | GTGCTCAAGGATTGAGCACTGAGCTACAGTGCTTCATCTCAGTGCTCAAGGATTGAGCAC(Cy5) | (SEQ ID NO:130) |
| mir-145 | GTGCTCAAGGATTGAGCACAAGGGATTCCTGGGAAAACTGGACGTGCTCAAGGATTGAGCAC(Cy5) | (SEQ ID NO:131) |
| mir-196 | GTGCTCAAGGATTGAGCACCCAACAACATGAAACTACCTAGTGCTCAAGGATTGAGCAC(Cy5) | (SEQ ID NO:132) |
| mir-216 | GTGCTCAAGGATTGAGCACCACAGTTGCCAGCTGAGATTAGTGCTCAAGGATTGAGCAC(Cy5) | (SEQ ID NO:133) |

We claim:

1. A detector probe comprising (a) a target-complementary portion, (b) a reporter group, and (c) a first stem-loop structure, wherein (i) the first stem-loop structure comprises a first segment, a second segment, and a third segment, (ii) the second segment is located between the first segment and the third segment, and (iii) the first segment and the third segment are complementary with each other.

2. The detector probe of claim 1, further comprising (d) a second stem-loop structure, wherein (i) the second stem-loop structure comprises a fourth segment, a fifth segment, and a sixth segment, (ii) the fifth segment is located between the fourth segment and the sixth segment, and (iii) the fourth segment and the sixth segment are complementary with each other.

3. The detector probe of claim 2, wherein the reporter group comprises a tether and an intercalating dye molecule.

4. The detector probe of claim 2, wherein the target-complementary portion further comprises a minor groove binder.

5. The detector probe of claim 1, wherein the reporter group comprises a tether and an intercalating dye molecule.

6. The detector probe of claim 1, wherein the target-complementary portion further comprises a minor groove binder.

7. A bimolecular detector probe comprising, (a) a first component comprising (i) a first target-complementary portion and (ii) a first reporter group and (b) a second component comprising (i) a second target-complementary portion and (ii) a second reporter group.

8. A detector complex comprising: (1) a detector probe comprising (a) a target-complementary portion, (b) a reporter group, and (c) a first stem-loop structure, wherein (i) the first stem-loop structure comprises a first segment, a second segment, and a third segment, (ii) the second segment is located between the first segment and the third segment, and (iii) the first segment and the third segment are complementary with each other; and (2) a displaceable sequence comprising a second reporter group, wherein the displaceable sequence is annealed to the detector probe.

9. The detector complex of claim 8, wherein the detector probe further comprises: (d) a second stem-loop structure, wherein (i) the second stem-loop structure comprises a fourth segment, a fifth segment, and a sixth segment, (ii) the fifth segment is located between the fourth segment and the sixth segment, and (iii) the fourth segment and the sixth segment are complementary with each other.

10. A method for detecting a small nucleic acid sequence comprising:
combining a detector complex with a sample, wherein the detector complex comprises (a) a detector probe comprising (i) a target-complementary portion and (ii) a first reporter group and (b) a displaceable sequence comprising a second reporter group, and wherein the detector probe is annealed with the displaceable sequence;
displacing the displaceable sequence of the detector complex and annealing a small nucleic acid sequence with the detector probe to form a duplex; and
detecting the detector probe-small nucleic acid sequence duplex.

11. The method of claim 10, wherein the first reporter group is a fluorophore and the second reporter group is a fluorescent quencher or a dark quencher.

12. The method of claim 10, wherein the detecting comprises quantitating the detector probe-small nucleic acid sequence duplex.

13. The method of claim 10, wherein the detecting comprises fluorescence microscopy.

14. The method of claim 10, wherein the detecting comprises single molecule detection.

15. The method of claim 10, wherein the sample comprises a cell or a tissue section.

16. The method of claim 10, wherein the sample comprises a cell lysate.

17. A method for detecting a small nucleic acid sequence comprising:
combining a detector probe with a sample, wherein the detector probe comprises (a) a target-complementary portion, (b) a reporter group, and (c) a first stem-loop structure, wherein (i) the first stem-loop structure comprises a first segment, a second segment, and a third segment, (ii) the second segment is located between the first segment and the third segment, and (iii) the first segment and the third segment are complementary, and wherein the reporter group comprises a tether and an intercalating dye molecule;
annealing the small nucleic acid sequence with the detector probe to form a detector probe-small nucleic acid sequence duplex; and
detecting the detector probe-small nucleic acid sequence duplex.

18. The method of claim 17, wherein the detecting comprises quantitating the detector probe-small nucleic acid sequence duplex.

19. The method of claim 18, wherein the quantitating comprises single molecule detection.

20. The method of claim 17, wherein the detecting comprises fluorescence microscopy.

21. The method of claim 17, wherein the sample comprises a cell or a tissue section.

22. The method of claim 17, wherein the sample comprises a cell lysate.

23. The method of claim 17, wherein the detector probe further comprises: (d) a second stem-loop structure, wherein (i) the second stem-loop structure comprises a fourth segment, a fifth segment, and a sixth segment, (ii) the fifth segment is located between the fourth segment and the sixth segment, and (iii) the fourth segment and the sixth segment are complementary with each other.

24. The method of claim 23, wherein the detecting comprises quantitating the detector probe-small nucleic acid sequence duplex.

25. The method of claim 24, wherein the quantitating comprises single molecule detection.

26. The method of claim 23, wherein the detecting comprises fluorescence microscopy.

27. The method of claim 23, wherein the sample comprises a cell or a tissue section.

28. The method of claim 23, wherein the sample comprises a cell lysate.

29. A method for detecting a small nucleic acid sequence comprising:
combining a bimolecular detector probe with a sample, wherein the bimolecular detector probe comprises, (a) a first component comprising (i) a first target-complementary portion and (ii) a first reporter group and (b) a second component comprising (i) a second target-complementary portion and (ii) a second reporter group;
annealing the small nucleic acid sequence with the two components of the bimolecular probe to form a trimolecular complex; and
detecting the trimolecular complex.

30. The method of claim 29, wherein the detecting comprises quantitating the trimolecular complex.

31. The method of claim 30, wherein the quantitating comprises single molecule detection.

32. The method of claim 29, wherein the detecting comprises fluorescence microscopy.

33. The method of claim 29, wherein the sample comprises a cell or a tissue section.

34. The method of claim 29, wherein the sample comprises a cell lysate.

35. A kit comprising a detector complex comprising a detector probe and a displaceable sequence.

36. The kit of claim 35, wherein the detector probe comprises: (a) a target-complementary portion, (b) a reporter group, and (c) a first stem-loop structure, wherein (i) the first stem-loop structure comprises a first segment, a second segment, and a third segment, (ii) the second segment is located between the first segment and the third segment, and (iii) the first segment and the third segment are complementary with each other.

37. The kit of claim 35, wherein the detector probe comprises: (a) a target-complementary portion, (b) a reporter group, (c) a first stem-loop structure, wherein (i) the first stem-loop structure comprises a first segment, a second segment, and a third segment, (ii) the second segment is located between the first segment and the third segment, and (iii) the first segment and the third segment are complementary with each other, and (d) a second stem-loop structure, wherein (i) the second stem-loop structure comprises a fourth segment, a fifth segment, and a sixth segment, (ii) the fifth segment is located between the fourth segment and the sixth segment, and (iii) the fourth segment and the sixth segment are complementary with each other.

38. A kit comprising a detector probe that comprises: (a) a target-complementary portion, (b) a reporter group, and (c) a first stem-loop structure, wherein (i) the first stem-loop structure comprises a first segment, a second segment, and a third segment, (ii) the second segment is located between the first segment and the third segment, and (iii) the first segment and the third segment are complementary with each other, wherein the reporter group comprises a tether and an intercalating dye molecule.

39. The kit of claim 38, wherein the detector probe further comprises: (d) a second stem-loop structure, wherein (i) the second stem-loop structure comprises a fourth segment, a fifth segment, and a sixth segment, (ii) the fifth segment is located between the fourth segment and the sixth segment, and (iii) the fourth segment and the sixth segment are complementary with each other

40. A kit comprising a bimolecular detector probe comprising: (a) a first component comprising (i) a first target-complementary portion and (ii) a first reporter group; and (b) a second component comprising (i) a second target-complementary portion and (ii) a second reporter group.

41. The kit of claim 40, wherein the first reporter group comprises a fluorescent reporter group and the second reporter group comprises a fluorescent quencher.
